(19) United States
(12) Patent Application Publication
(10) Pub. No.: US 2017/0042643 A1
(43) Pub. Date: Feb. 16, 2017

(54) SURFACE MODIFICATION FOR ENHANCED BONDING OF CERAMIC MATERIALS

(71) Applicant: RESEARCH TRIANGLE INSTITUTE, Research Triangle Park, NC (US)

(72) Inventors: Jeffrey Robert Piascik, Raleigh, NC (US); Brian R. Stoner, Chapel Hill, NC (US)

(73) Assignee: RESEARCH TRIANGLE INSTITUTE

(21) Appl. No.: 15/292,289

(22) Filed: Oct. 13, 2016

Related U.S. Application Data
(62) Division of application No. 13/880,277, filed on Apr. 18, 2013, now abandoned.
(60) Provisional application No. 61/394,986, filed on Oct. 20, 2010.

(51) Int. Cl. A61C 8/00; A61C 5/04; A61C 5/10; A61C 7/14; A61C 13/00; B05D 3/14; B05D 3/10; B05D 3/04; A61K 6/02; A61K 6/08; A61L 27/50; A61L 27/14; A61L 27/10; A61L 27/06; A61C 5/02

(52) U.S. Cl. CPC A61C 8/0015; A61C 5/02; A61C 5/04; A61C 5/10; A61C 7/14; A61C 13/0006; B05D 3/141; B05D 3/104; B05D 3/0433; A61K 6/024; A61K 6/0235; A61K 6/0225; A61K 6/0205; A61K 6/08; A61L 27/50; A61L 27/14; A61L 27/105; A61L 27/10; A61L 27/06; A61L 2400/18; A61L 2430/12

(57) ABSTRACT

A fluoride treated medical implant, such as a dental component, is provided, the medical implant comprising fluorinated metal oxide on the substrate surface. A method for the preparation of such treated implants is also provided, the method involving exposure of the medical implant to a fluorine-containing reagent. A dental structure is also provided, which includes a first dental component comprising a fluorinated metal oxide layer on its surface, a silane coupling agent, a dental cement, and a second dental component having a surface bonded to the dental cement. An additional dental structure, which includes a first dental component comprising a fluorinated metal oxide layer on its surface, a dental cement, and a second dental component having a surface bonded to the dental cement is also provided.

### FIELD OF THE INVENTION

The invention is related to methods for affixing medical implants, including dental and orthopedic implants and devices, by functionalizing the surface of the implants or devices. It is also related to medical implants wherein the outer surface may be functionalized to afford reactivity with various other materials.

### BACKGROUND OF THE INVENTION

Statistics show that nearly 70% of adults ages 35 to 44 have lost at least one permanent tooth to an accident, gum disease, a failed root canal, or tooth decay. By age 74, it is reported that 26% of adults have lost all of their permanent teeth. Both the increasing aging population and a growing awareness for oral health and aesthetics have led to the growth of dental implant surgery. A dental implant is a permanent post anchored to the jawbone and topped with a prosthetic (implant abutment and synthetic crown or bridge) that can be permanently attached to the post. Single teeth or an entire arch of teeth may be effectively replaced with dental implants and attached prosthetics, which can last for significant periods of time with routine maintenance. Dental implant surgery is now considered to be the fastest growing area in dentistry.

Dental implant posts are typically made of titanium or titanium alloys, which generally are anchored to bone via osseointegration (intimate physical contact between the synthetic implant and the surrounding bone). Traditionally, metallic prosthetic components have been used to restore implants. However, recent commercial development has focused on alternative materials, especially ceramics. Ceramics provide high strength as well as the natural look of real teeth. In many cases, ceramics have higher wear resistance, corrosion resistance, toughness, and strength than metals and metal alloys. In particular, recent research has focused on high strength ceramics such as alumina and zirconia. These materials provide better fracture resistance and long-term durability than traditional porcelain and other ceramics.

The methods for attaching a substrate (natural tissue like tooth structure or implant abutment) to a prosthetic restorative may be micromechanical, or may additionally include chemical bonding through silanation or other surface treatment techniques. In some applications, adhesive bonding is not required and the ceramic material may be placed and affixed using conventional cements that rely on micromechanical retention. Micromechanical retention may be achieved in some cases by merely roughening the surfaces of the substrate or the restorative. However, these conventional cementation techniques do not provide the high bond strength required for some applications. In such applications, good adhesion is often important for high retention, prevention of microleakage, and increased fracture and fatigue resistance, and may be provided by resin-based cements used in conjunction with intermediate adhesion promoters, like dental silanes. Strong resin bonding relies on micromechanical interlocking as well as adhesive chemical bonding to the ceramic surface and requires a combination of surface roughening and chemical functionalization for efficient attachment.

Surface roughening may be achieved by grinding, abrasion with diamond rotary instruments, surface abrasion with alumina particles, acid etching with acids such as hydrofluoric acid (HF), or a combination of these techniques. Adhesive chemical bonding is commonly achieved through a two-step process, which initially involves treating the implant or restorative with a silane coupling agent. Silane coupling agents are organic compounds that contain silicon atoms, are similar to orthoesters in structure, and may display dual reactivity. Silanes typically contain one or more alkoxy groups, wherein the alkoxy groups can react with an inorganic substrate. The other end of the molecule is organically functionalized, for example, with a vinyl, allyl, isocyanate, or amino group, and can polymerize with an organic matrix such as a methacrylate. The next step of achieving the adhesive chemical bonding is using an organic resin-based cement to react with the organically functionalized silane to affix adherends.

This adhesive chemical bonding, which is required for many dental applications, is not applicable to high strength ceramic materials. Because of the composition and physical properties of high-strength ceramics, they are not easily etched or chemically functionalized using conventional treatments. Traditional silane chemistry is not effective with high strength ceramics because such materials are more chemically stable (inert) than silica-containing materials and are not as easily hydrolyzed. Furthermore, due to their hardness and strength, the surfaces of high strength ceramics are not easily roughened. Acid etchants such as HF do not sufficiently roughen the surface. These materials may be roughened only by very aggressive mechanical abrasion methods, which may create fatigue-enhancing surface flaws.

One method that can be used to provide adhesive chemical bonding of high strength ceramics requires surface abrasion with alumina particles coated with silica. The alumina particles impact the surface, transferring a thin silica layer via a tribochemical process, which allows for chemical bonding to a silane coupling agent, which can then bond to a resin-based cement. However, this method is a relatively complicated procedure and does not produce bond strengths as high as those reported for silane-bonded porcelain. In addition, air particle abrasion may be particularly unsuitable for zirconia-based materials, as it is likely to generate micro-fractures which could lead to premature, catastrophic failure.

Alternatively, the use of phosphoric acid primers or phosphate-modified resin cements has been shown to produce silane-like adhesion through similar types of hydrolyzation-driven chemistry.

However, the bond strengths reported are generally even lower than those reported for the tribochemical silica coating in combination with silane and resin cement. One recent study has shown increased bond strength using selective infiltration etching and novel silane-based zirconia primers. See Aboushelib M N, Matinlinna J P, Salameh Z, Ounsi H., Innovations in Bonding Zirconia-Based Materials: Part I. Dent. Mat. 2008; 24: 1268-1272. However, the available approaches for adhesive bonding of high strength ceramics are not adequate for all clinical applications and their long-term efficacy is currently unknown.

Another recent study has demonstrated that silanation of ceramic surfaces by molecular vapor deposition may afford a useful strategy for the preparation of coated materials, which may be further functionalized using silane coupling agents and traditional dental cements. See U.S. application Ser. No. 13/273,528, filed Oct. 14, 2011 and International Application No. PCT/US10/31348, both to Piascik et al., and both incorporated herein by reference in their entireties. However, this method may not be readily useful in the clinic due to the necessity of specialized molecular vapor deposition equipment. Furthermore, in order to provide surfaces suitable for use with existing adhesive bonding techniques, it may be beneficial to have a surface preparation method rather than a coating method to afford a material with fewer surface interfaces.

### BRIEF SUMMARY OF THE INVENTION

In one aspect of the present invention is provided a medical implant comprising a substrate surface comprising a fluorinated metal oxide. The substrate surface of the medical implant may comprise, for example, zirconia, alumina, titania, chromium oxide, or a combination thereof. In some embodiments, the fluorinated metal oxide comprises a mixture of metal oxyfluoride and metal fluoride phases. In certain embodiments, the fluorinated metal oxide is from about 0.5 nm to about 5 nm thick. The structure and purpose of the medical implant can vary. In some embodiments, the medical component comprises one or more dental components including, but not limited to, a dental implant, crown, bridge, filling, veneer, inlay, onlay, endodontic device, or orthodontic bracket.

In some embodiments, the medical implant further comprises a silane coupling agent overlying the fluorinated metal oxide. The silane coupling agent may be, for example, 3-methacryloyloxypropyltrimethoxysilane, 3-trimethoxysilylpropylmethacrylate, 3-acryloyloxypropyltrimethoxysilane, 3-isocyanatopropyltriethoxysilane, N-[3-(trimethoxysilyl)propylethylenediamine], 3-mercaptopropyltrimethoxysilane, bis-[3-(triethoxysilyl)propyl]polysulfide, or a combination thereof.

The medical implant can, in certain embodiments, further comprise a dental cement overlying the medical implant or overlying the silane coupling agent. In some embodiments, the dental cement is a polymer-based adhesive, cement or composite, resin-modified glass ionomer, or a combination thereof. The overlying dental cement can be covalently bonded to the silane coupling agent. In an alternative embodiment, the overlying dental cement can be overlying and coupled to the fluorinated metal oxide with no silane coupling agent therebetween.

In one specific aspect, the medical implant comprises a first dental component comprising a metal oxide and having a substrate surface comprising the fluorinated metal oxide; an optional silane coupling agent overlying the fluorinated metal oxide; a dental cement overlying the fluorinated metal oxide or optional silane coupling agent; and a second dental component having a surface bonded to the dental cement. In such embodiments, the second dental component can vary; for example, in some embodiments, the second dental component is selected from the group consisting of a dental implant, crown, bridge, filling, veneer, inlay, onlay, endodontic device, or orthodontic bracket. The material comprising the second dental component can also vary and can be, for example, natural tooth, metal, porcelain fused to metal, porcelain, ceramic, resin, or a combination thereof.

In some embodiments, the medical implant can be characterized by a surface comprising fluorinated metal oxide with a contact angle of less than about 25° or less than about 10°.

In another aspect of the invention is provided a method of preparing the surface of a medical implant, comprising the steps of providing a medical implant comprising a substrate surface formed of a material comprising available hydroxyl groups; and treating the medical implant with a fluorine-containing reagent to provide a fluorinated metal oxide on the implant surface. The method can, in certain embodiments, further comprise reacting the fluorinated metal oxide surface with a silane coupling agent.

In some embodiments, the method further comprises coupling the silane coupling agent to a dental cement. In other embodiments, the method comprises reacting the fluorinated metal oxide surface with a dental cement with no silane coupling agent therebetween. As described above, the nature of the silane coupling agent, and the dental cement can vary.

In some embodiments, the dental cement is used to bond a dental component to a second dental component. The dental components can be, for example, selected from dental implants, crowns, bridges, fillings, veneers, inlays, onlays, endodontic devices, or orthodontic brackets. The surface of the second dental component can be, for example, natural tooth, metal, porcelain fused to metal, porcelain, ceramic, resin, or a combination thereof.

In some embodiments, the treating step comprises plasma treatment. In some other embodiments, the treating step comprises physical roughening or chemical etching of the surface prior to or at the same time as treating the implant with the fluorine-containing reagent. In certain embodiments, the fluorine-containing reagent is sulfur hexafluoride (SF₆).

### BRIEF DESCRIPTION OF THE SEVERAL VIEWS OF THE DRAWINGS

Having thus described the invention in general terms, reference will now be made to the accompanying drawings, which are not necessarily drawn to scale, and wherein:

FIG. 1 is a schematic representation of the plasma surface modification process on a zirconia surface;

FIG. 2 is a graph showing shear bond stress values for various modified and unmodified zirconia surfaces;

FIGS. 3a and 3b are SEM micrographs at different magnifications of a fluorinated (polished) surface with an adhesive/cohesive failure mode; FIGS. 4a and 4b are XPS core scans of the Zr 3d doublet of an untreated zirconia sample (FIG. 4a) and a fluorinated zirconia sample (FIG. 4b);

FIG. 5 is a graph depicting the relationship between Zr 3d binding energy and the Pauling charge on Zr⁺-cation, with added data points of Zr 3d binding energies measured via XPS from untreated and fluorinated zirconia specimens;

FIG. 6 is a schematic of fluorinated plasma (SF₆ as source gas) induced phase conversion on an yttrium-stabilized zirconia surface;

FIG. 7 is a graph showing shear bond strength values for various modified and unmodified yttrium-stabilized zirconia surfaces;

FIGS. 8a, 8b, and 8c are scanning electron micrographs at different magnifications of a 2 min (roughened) fluorinated specimen failure surface (dark regions are resin cement and white regions are yttria-stabilized zirconia surface);

FIGS. 9A and 9B show XPS analysis of the Zr-3d (FIG. 9A) and the Y-3d spectra (FIG. 9B) as a function of fluorination plasma treatment time;

FIG. 10a is a graph showing the relationship of shear bond strength and change in Y-surface concentration as a function of plasma treatment time;

FIGS. 10b and 10c are contact angle images of a 2-minute treated and an untreated control specimen, respectively;

FIGS. 11A and 11B are X-ray diffraction scans of bulk material (FIG. 11A) and 2° glancing angle (FIG. 11B); and

FIG. 12 is a plot of Y/Zr concentration versus time comparing the relative yttrium to zirconium levels and respective bonding components for XPS deconvolution.

### DETAILED DESCRIPTION OF THE INVENTION

The present invention now will be described more fully hereinafter with reference to the accompanying drawings, in which some, but not all embodiments of the inventions are shown. Indeed, these inventions may be embodied in many different forms and should not be construed as limited to the embodiments set forth herein; rather, these embodiments are provided so that this disclosure will satisfy applicable legal requirements. Like numbers refer to like elements throughout. As used in the specification, and in the appended claims, the singular forms “a”, “an”, “the”, include plural referents unless the context clearly dictates otherwise.

One aspect of the invention relates to methods of preparing the surface of a medical implant (which may be a dental or orthopedic implant or device) for further functionalization. In certain embodiments, the method relates to using fluoride treatment to prepare the implant surface. Preparing the surface of a medical implant in this way allows the implant to be subsequently silanated and/or affixed to a variety of surfaces using conventional cements or resins. Another aspect of the invention relates to fluoride-treated medical implants. A further aspect relates to subsequently silanated medical implants and to silanated medical implants further reacted with one or more cements or resins, which can be used to affix the medical implants to a variety of surfaces. Another aspect of the invention relates to fluoride-treated medical implants that are directly reacted with one or more cements or resins, which can be used to affix the medical implants to a variety of surfaces.

“Medical implant” as used herein means any physical object that can be implanted into the body or which comes in direct contact with the body. Medical implants that may be used according to the methods of the present invention include, but are not limited to, dental components, including dental implants, restoratives, and orthodontic devices, as well as orthopedic devices and implants. Any medical implant that may be affixed to another surface or device by a resin or cement may be surface-treated according to the present invention.

The medical implant can comprise any surface material comprising available hydroxyl groups on its surface. For example, the medical implant may be a metal, which inherently has a metal oxide layer on its surface, a polymer or copolymer, or a metal oxide. In certain embodiments, the metal implant comprises a refractory metal oxide. In one embodiment, the medical implant comprises a ceramic material. In some embodiments, the medical implant may comprise zirconia, alumina, titania, or chromium-oxide-based material or a combination thereof. In certain embodiments wherein the medical implant comprises a ceramic, the ceramic may be unstabilized (i.e., pure) or may comprise a stabilized material, e.g., a fully or partially stabilized ceramic material. For example, in specific embodiments, the ceramic may be stabilized with an oxide (e.g., yttrium oxide, magnesium oxide, calcium oxide, and/or cerium(III) oxide). In certain specific embodiments, the medical implant comprises yttria-stabilized zirconia (YSZ). In another embodiment, the medical implant may be a metallic device that is surface passivated with an oxide film. For example, the surface of the metal implant may comprise titanium oxide on a titanium alloy, or chromium oxide on stainless steel or cobalt chrome.

“Dental implant” as used herein means a post (i.e., a dental abutment) anchored to the jawbone and topped with individual replacement teeth or a bridge that is attached to the post or posts. The term is meant to encompass traditional dental implants as well as mini-dental implants. In some cases where the dental abutment is in the form of natural tooth, the dental implant only comprises the implanted replacement tooth or bridge.

“Restorative” as used herein means any dental component used to restore the function, integrity and/or morphology of any missing tooth structure. Examples of restoratives that may be coated according to the methods described herein include, but are not limited to, crowns, bridges, fillings, veneers, inlays and onlays, as well as endodontic devices including endodontic cones and devices for endodontic root perforation repair.

“Orthodontic device” as used herein means any device intended to prevent and/or correct irregularities of the teeth, particularly spacing of the teeth. Orthodontic devices particularly relevant to the present invention include but are not limited to orthodontic brackets.

“Dental component” as used herein encompasses any component of a dental implant or a restorative or an orthodontic device and can even include, in certain embodiments, natural tooth.

“Orthopedic device” or “orthopedic implant” as used herein means a device that replaces a part or function of the body. Orthopedic devices include but are not limited to devices adapted to form artificial joints, including hips, knees, and elbows.

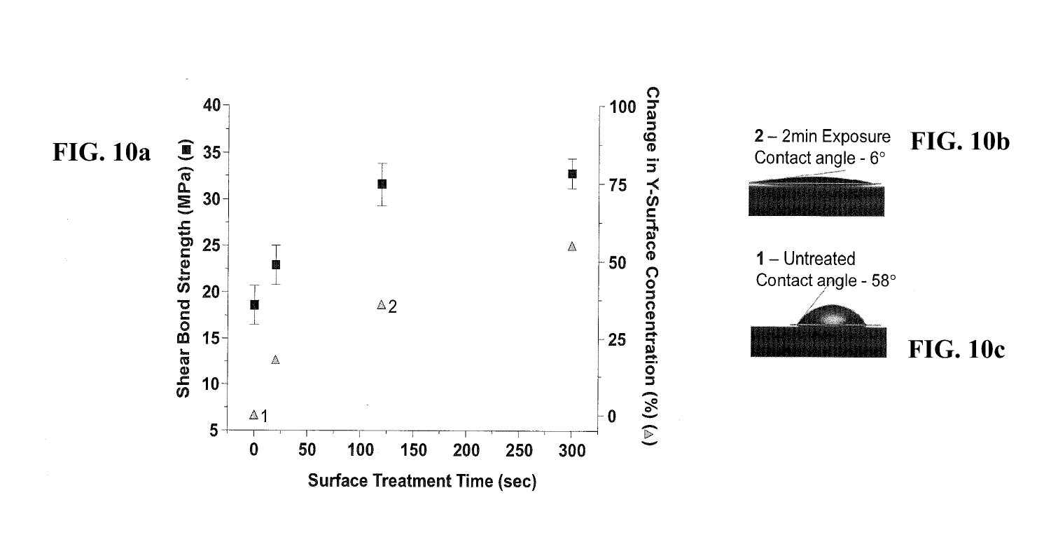

In one aspect of the present invention is provided a method for fluoride treatment of the surface of the medical implant by exposing the medical implant to a fluorine-containing reagent. In some embodiments, such treatment changes the chemical makeup of the surface of the medical implant. In certain embodiments, the fluoride treatment of the surface provides a surface that is more reactive than the untreated surface. Thus, this method may provide a surface that is more susceptible to further functionalization with various reagents. Although not bound by any theory of operation, it is believed that the fluorination processes of the invention result in fluorine replacing oxygen in the oxide lattice near the surface of the medical implant, thus creating a metastable, partially covalent, partially ionic bond capable of reacting with conventional silane coupling agents and, in certain embodiments, even capable of directly reacting with conventional dental cements without the need for an intervening silane coupling agent. In certain embodiments, the fluoride treatment of the surface as disclosed herein provides a surface with higher wettability than the untreated surface. Interestingly, fluoride treatment is typically conducted to make a surface more hydrophobic. However, as disclosed herein, in certain embodiments, fluoride treatment may provide a surface characterized by a higher wettability (i.e., greater hydrophilicity) than the untreated surface. See, for example, FIGS. 10b and 10c. In some embodiments, this higher wettability may be quantified by a smaller contact angle than that observed prior to fluoride treatment. For example, in certain embodiments, the contact angle may be less than about 50°, less than about 25°, less than about 10°, or less than about 8°. In certain embodiments, the contact angle may be between about 5° and about 20°, or between about 5° and about 10°. The contact angle may be determined with any method typically used for this purpose. For example, in some specific embodiments, a KRUSS EasyDrop Standard instrument is used. In certain embodiments, AS™ D7490 (2008) is used to determine wettability.

In some embodiments, the fluoride treatment is accomplished by plasma treatment. Plasma treatment, as used herein, generally comprises exposing the medical implant to a fluoride ion source in plasma form. Typically, such a method involves generating a plasma field in an electrically charged atmosphere, e.g., in a plasma chamber. A traditional plasma setup comprises a chamber in which the sample to be treated may be contained, which is capable of receiving a selected gas flow; a vacuum source; and a power supply. However, any setup capable of providing fluoride ions in plasma form may be used according to the presently disclosed method. For example, in one specific embodiment, a planar inductively coupled RF Plasma tool from Oxford Instruments may be used.

In some embodiments, exposure to plasma treatment allows low molecular weight materials such as water and adsorbed gases to be removed from the surface to expose a clean, fresh surface. Some percentage of the reactive components in the plasma have sufficient energy bond to the freshly exposed surface, changing the chemistry of the surface and imparting the desired functionalities. In certain embodiments, the reactive components comprise fluoride ions.

The composition of the plasma may be varied. The fluoride ion source may be any fluorine-containing reagent in gas or liquid form that, in plasma form, can provide fluoride ions. For example, in some embodiments, the fluoride ion source comprises sulfur hexafluoride (SF₆). In other embodiments, the fluoride ion source comprises CF₄, C₄F₈, C₅F₈ (octafluorocyclopentene), C₄F₆ (hexafluoro-1,3-butadiene), NF₃, SiF₄, or combinations thereof. In some embodiments, the fluoride ion source comprises a chlorofluorocarbon (CFC). A CFC is any compound having chlorine, fluorine, and carbon atoms. For example, when derived from methane and ethane, CFCs have the formulae CClₘF₄₋ₘ and C₂ClₘF₆₋ₘ respectively, where m is nonzero. In some embodiments, the fluoride ion source comprises a hydrofluorocarbon (HFC). An HFC is any compound having hydrogen, fluorine, and carbon atoms. For example, when derived from methane, ethane, propane, and butane, these compounds have the formulae CFₘH₄₋ₘ, C₃FₘH₈₋ₘ, and C₄FₘH₁₀₋ₘ respectively, where m is nonzero. In some embodiments, the fluoride ion source comprises a hydrochlorofluorocarbon (HCFC). An HCFC is any compound having hydrogen, chlorine, fluorine, and carbon atoms. For example, when derived from methane and ethane, HCFCs have the formulae CClₘFₘH₄₋ₘ₋ₙ and C₂ClₓF_{y}H_{6-x-y} respectively, where m, n, x, and y are nonzero. In some embodiments, the fluoride ion source comprises a bromochlorofluorocarbon or bromofluorocarbon. These compounds are similar to HCFCs and CFCs, respectively, with bromine atoms in place of the chlorine atoms.

This list of fluoride ion source reagents is not intended to be limiting. Other liquid or gaseous reagents capable of providing fluoride ions in plasma form are also contemplated as being useful according to the presently described method.

The parameters within the plasma chamber may vary. For example, the power source used to generate the plasma may be of any type, including but not limited to, DC, RF and microwave. The electrode configuration used to generate the plasma may also be varied. The degree of ionization within the plasma may be varied, including fully ionized, partially ionized, or weakly ionized. The pressure at which the system operates may be varied, including but not limited to, within the range of vacuum pressure (<10 mTorr or 1 Pa) to moderate pressure (˜1 Torr or 100 Pa) to atmospheric pressure (760 Torr or 100 kPa). The temperature relationships within the plasma may also be varied, ranging from a thermal plasma (Tₑ=Tᵢₒₙ=T_{gas}), where e=electron, to a non-thermal or “cold” plasma (Tₑ>>Tᵢₒₙ=T_{gas}). The plasma may be magnetized, partially magnetized, or non-magnetized.

The period of time for which the medical implant is exposed to the plasma may vary. In certain embodiments, the exposure time ranges from about 1 second to about 100 minutes, and preferably from about 20 seconds to about 2 minutes. The plasma power may vary. In certain embodiments, the plasma power is within the range of about 50 to about 1000 W, and preferably within the range of about 600 to about 800 W. One specific set of parameters that may be used according to the present invention includes a planar, inductively coupled 13.56 MHz radio-frequency plasma reactor at 800 W with a dc bias of ˜300V.

The substrate may be untreated or may be treated in some way prior to being subjected to the fluoride treatment. For example, the surface of the substrate may be roughened, for example, by polishing with polishing paper, and/or air-abrading with alumina or other types of particles. The degree of surface roughening required may vary, depending on the particular application. The substrate may be treated with oxygen-containing plasmas prior to the disclosed treatment method, for example, to eliminate organic contaminants.

Although the fluoride treatment method described above relates to plasma treatment, other means for the fluoride treatment of a medical implant surface are contemplated and encompassed within the present invention. In some embodiments, any liquid reagent capable of generating fluoride ions may be used in combination with a physical or chemical treatment capable of providing sufficient energy to facilitate reaction between the surface and the fluoride ions in the absence of plasma generation. In this manner, treatment of the surface could occur using a slurry or gel comprising the fluoride ion source in combination with a second component that provides physical roughening or chemical etching of the surface. The role of the second component is to facilitate scission of bonds in the metal oxide structure, resulting in enhanced reactivity of the metal oxide surface with the fluorine-containing reagent. In some embodiments, the fluoride ion source and the second component are provided within the same composition. In some embodiments, the fluoride ion source and the second component are provided within separation compositions. In some embodiments, the fluoride ion source and the second component are applied to the medical implant together. In other embodiments, the fluoride ion source and the second component are applied as separate treatments (e.g., a physical or chemical treatment is first applied to the implant, followed by treatment with a composition comprising a fluoride ion source).

For example, in certain embodiments, the medical implant is treated with a reactive chemical etchant in combination with a fluoride-generating reagent. In some embodiments, the reactive etchant and/or fluoride-generating reagent may be contained within a solution or slurry, including, but not limited to, an aqueous solution. The reactive etchant may be any reagent that etches the surface of the medical implant. For example, the etchant may comprise sulfuric acid (H₂SO₄), hydrofluoric acid (HF), hydrochloric acid (HCl), hydrogen peroxide (H₂O₂), phosphoric acid (H₃PO₄), ferric chloride (FeCl), nitric acid (HNO₃), or a combination thereof. For example, in certain embodiments, the etchant may comprise a combination of HF and HNO₃ or H₂SO₄ and HNO₃. Obviously, the composition of the medical implant will govern which reagents will etch the surface of the implant. Other reagents that may etch the material comprising the medical implant are also encompassed within the class of reagents that may be used for this purpose.

In certain embodiments, the medical implant is treated with a physical abrasive and reacted with a fluoride-containing reagent. In some embodiments, the physical abrasive may be contained within a gel-type composition. The physical abrasive may be any material that can roughen the surface of the medical implant. For example, the physical abrasive may be pumice, diamond grit, alumina or zirconia particles, and/or silicon carbide. Other materials that may physically roughen the medical implant surface are also encompassed within the class of physical abrasives that may be used for this purpose. In some embodiments, a combination of chemical etchant and mechanical abrasive may be used.

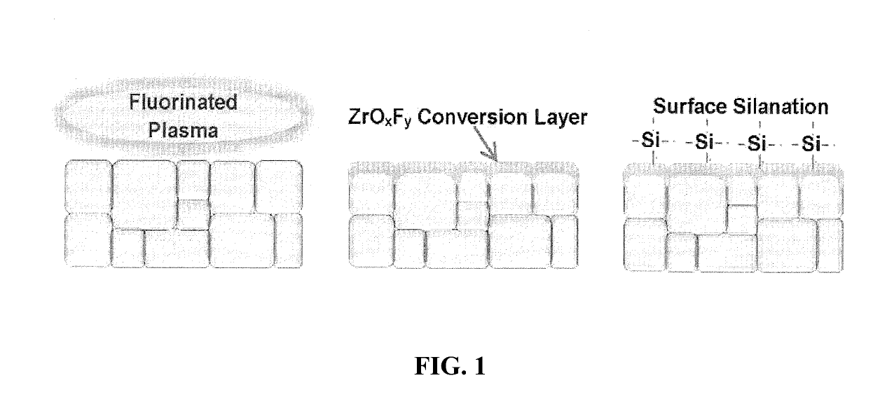

In some embodiments, the method further comprises applying a silane coupling agent to the medical implant following the preparation of the surface (i.e., after the surface fluorination process described above). For example, in some embodiments, this method is shown generally in FIG. 1. By “silane” or “silane coupling agent” as used herein is meant any compound containing one or more silicon (Si) atoms. Silanes resemble orthoesters, and can be bifunctional. The silanes useful for the present invention are typically bifunctional with dual reactivity. In particular, they are typically able to react with an inorganic substrate and with an organic matrix. Such silanes may include one or more organic functionalities, including but not limited to vinyl, allyl, amino, or isocyanato groups. They also typically contain one or more alkoxy groups, including but not limited to methoxy and ethoxy groups. Silanes may contain one or more other substituents, which may be reactive, including chloride. There may also be an alkyl or alkylene link between the Si and the organic functionality.

Silanes may be hydrophilic or hydrophobic, and can also be anionic or cationic. In some embodiments, the silanes are trialkoxysilanes, with three alkoxy groups and one organic functionality. The silanes useful in the present invention include but are not limited to 3-methacryloyloxypropyltrimethoxysilane, 3-trimethoxysilylpropylmethacrylate, 3-acryloyloxypropyltrimethoxysilane, 3-isocyanatopropyltriethoxysilane, N-[3-(trimethoxysilyl)propylethylenediamine], 3-mercaptopropyltrimethoxysilane, and bis-[3-(triethoxysilyl)propyl]polysulfide. There are many silanes that are commercially available. Examples include RelyX™ Ceramic Primer, Monobond™-S, Fusion,™ Vectris™ Wetting Agent, Porcelain Repair Primer, Pulpdent™ Silane Bond Enhancer, Silanator,™ Cerinate® Primer, Silicoup™ A and B, Ultradent™ Porcelain Etch & Silane, Clearfil™ Porcelain Bond Activator, Clearfil™ Ceramic Primer, Prolong Silane Bond Enhancer, Quadrant™ Porcelain Coupling Agent, Bifix DC,™ Bisco™ Porcelain Primer, Cimara,™ and ESPE™ Sil.™ Exemplary manufacturers of such silanes include 3M/ESPE, Ivoclar Vivadent, Pulpdent Corporation, Bisco, Inc., Kurayray, Premier Products Company, Mirage, Ultradent Products, Inc., George Taub Products, Cosmedent, VOCO America, Inc., Cavex Holland BV, and Kerr Corporation.

Applying a silane to an inorganic surface typically involves hydrolysis and condensation reactions with the surface. The silane may be applied in polar aqueous alcohol solutions, ethyl acetate, nonpolar solutions, or mixtures thereof. For example, the solution may comprise an acetone/ethanol mixture. Preferably, the silane is applied in aqueous alcohol solutions, such as 90-95% ethanol or isopropanol, or more dilute aqueous alcohol solutions from about 20-50% ethanol or isopropanol. The OR groups of the silane may be hydrolyzed, becoming OH groups. The one or more alkoxy groups and/or OH groups on the silane may react with free hydroxyl groups on the surface of the inorganic material. The silanes may react with other silanes to form dimers (siloxanes), which may condense to form siloxane oligomers. Such reactions may result in branched hydrophobic siloxane bonds. The siloxane oligomers, siloxane monomers, and/or silanes may react with the inorganic material to form M—O—Si bonds, wherein M is any metal. In some embodiments, the substrate comprises a zirconia or alumina substrate which may be a medical implant.

The organic functional end of the silane may be used to polymerize with an organic matrix such as a dental or orthopedic cement. “Cement,” as used herein includes both traditional cements and resins, and refers to any adhesive material used to attach any synthetic or natural dental, orthodontic, or orthopedic implant or device to another substrate. Cements of particular interest herein are polymer-based cements, including methacrylate-based cements and include, but are not limited to, polymer-based adhesives, cements, and composites, and resin-modified glass ionomers. Exemplary dental cements which may be utilized in the present invention include, but are not limited to, the products identified by the tradenames Multilink® Universal Paste, Vivaglass® CEM, Appeal™ Esthetic Resin Cement, Variolink® Esthetic Resin Cement, Panavia,™ RelyX™ Unicem, RelyX™ Arc, Advance,™ Fuji Plus,™ Calibra,® Linkmax,™ Duolink,™ Integracem,™ Biscem,™ Imperva™ Dual, Contact Cure,™ Embrace,™ NX3 NEXUS® Cement, C&B Metabond,™ All-Bond,™ Geristore,® Vitique,® Permabond® Cyanloacrylate adhesive, and Superbond C&B.™ Exemplary manufacturers of such dental cements include, but are not limited to, Ivoclar Vivadent, Kuraray, Bisco, Inc., Kerr Corporation, Premier Products Company, Pulpdent Corporation, 3M/ESPE, Cosmedent, Dentsply International, GC America Inc., Parkell Inc., and Ultradent Products Inc.

The type of cement chosen may depend on the structure to which the medical implant is to be bonded. For example, a glass ionomer or zinc polycarboxylate cement is typically used to attach an implant or restorative to natural tooth. Other considerations in the selection of a dental cement include solubility, erosion, tensile strength, shear strength, toughness, elastic modulus, creep, working and setting time, sensitivity to moisture during and after setting, thermal conductivity and diffusivity, pH during setting, biocompatibility, compatibility with other restorative materials, potential for fluoride release, adhesion to enamel and dentine, sensitivity of setting reaction to temperature, rate of change in viscosity, film thickness, and dimensional change in the presence of moisture. Glass ionomer cements are capable of releasing fluoride, and may be particularly suitable in geriatric dentistry. The resin and resin-glass ionomer cements are stronger and tougher than the other cements. In one embodiment of the present invention, a resin-based cement is used.

The cements may contain various other additives. Some cements include ingredients to etch, prime, and/or bond. Some cements include components that are capable of releasing fluoride on a sustained basis. Cements may or may not be adhesive. For example, zinc phosphates are typically not adhesive, while resin-modified glass ionomers exhibit both chemical and mechanical adhesion.

The cements may be temporary or permanent, but preferably are permanent. The cement may be applied to the silane functionalized surface of the medical implant by any means known in the art. Cements are often sold as powders and are mixed with liquid prior to use and applied to the organo-functionalized silane surface and to the material to which the medical implant will be coupled. Variables affecting the cement and the success of the bonds formed include mixing time, humidity, powder to liquid ratio, and temperature. Alternatively, the cement may be sold and used as a paste. Some cements, such as polymer-based resins, require curing. Curing typically requires the use of light or chemical activation or may require both. Alternatively, some cements are self-curing.

In certain embodiments, the method comprises directly reacting the fluorinated surface of a medical implant with a cement. In other words, in certain embodiments, the silane coupling agent is unnecessary. In certain embodiments, the fluorinated surface of the implant is believed to be sufficiently reactive with conventional dental cements that adequate bond strengths can be obtained without requiring the additional step of reacting the surface with an intervening silane coupling agent.

The medical implant may be attached to various types of material using the cement. In certain embodiments, the cement is a resin cement. In some embodiments, the coated medical implant is a dental component, which may be bonded, for example, to any underlying substrate (e.g. tooth structure or implanted abutment). In other embodiments, the cement may be used to bond the treated medical implant to a ceramic, porcelain or metal material. In one embodiment, the treated medical implant is a dental implant, which is attached to a crown that may comprise any material, including a metal, porcelain fused to metal, porcelain, ceramic, or resin.

In certain embodiments, a fluoride-treated medical implant that has been treated with an organosilane and subsequently bonded to another surface with cement has a higher shear bond strength than medical implants that have not been fluoride-treated. Similarly, in certain embodiments, a fluoride-treated medical implant that has been directly reacted with cement exhibits higher shear bond strength than medical implants that have not been fluoride-treated.

In one embodiment of the present invention, the fluorination treatment described herein is followed by a molecular vapor deposition (MVD) process to apply a silicon oxide coating to the substrate. For a more detailed description of such a process, see, for example, U.S. application Ser. No. 13/273,528, filed Oct. 14, 2011 and International Application No. PCT/US10/31348, both to Piascik et al., and both incorporated herein by reference in their entireties. In such embodiments, the reagents utilized in the molecular vapor deposition include one or more silicon-based precursors. Briefly, a silicon-based precursor and optionally one or more additional reagents react with the surface, forming active hydroxyl groups on the surface, subsequently forming a silicon oxide layer on the substrate surface. The silicon-based precursor may be any silicon-containing species, including mono-, di-, and tri-silanes and siloxanes that can be vaporized. The silicon-based precursors include, but are not limited to, tetrachlorosilane (SiCl₄), tetrafluorosilane (SiF₄), tetrabromosilane (SiBr₄), trichlorosilane (HSiCl₃), trifluorosilane (HSiF₃), tribromosilane (HSiBr₃), hexachlorodisilane (Si₂Cl₆), hexachlorodisiloxane (Si₂Cl₆O), and combinations thereof. In one embodiment, the silicon-based precursor is tetrachlorosilane and an additional reagent is water vapor. In some embodiments, multiple layers are deposited on the medical implant via this method. The deposited silicon oxide layer may be continuous or discontinuous on the surface of the medical implant.

The surface fluorination process of the present invention, when used prior to and in combination with the MVD process described above, may lead to better performance of the surface as compared to a surface that may be obtained using MVD on a non-fluoride treated surface. In some aspects, the fluorination treatment may provide a more reactive surface, leading to a more effective silicon oxide-coated surface following MVD, such as by improving adhesion of the silicon oxide layer or improving surface coverage of the silicon oxide layer. A silicon oxide coating may be desirable in certain applications, including but not limited to, applications involving cell attachment and/or integration.

Another embodiment provides a medical implant that is treated according to the processes described above. The medical implant is preferably zirconia or alumina but may comprise any material which may have available hydroxyl groups on its surface. In some embodiments, the medical implant may comprise titania, or chromium oxide. In certain specific embodiments, the medical implant comprises yttria-stabilized zirconia (YSZ). The implant may have an activated surface resulting from treatment with a fluoride-containing reagent as described above. In certain embodiments, the substrate surface comprises a fluorinated metal oxide. Although not wishing to be bound by theory, it is thought that the activated surface comprises a metal oxyfluoride (e.g., zirconium oxyfluoride (ZrOₓF_{y})).

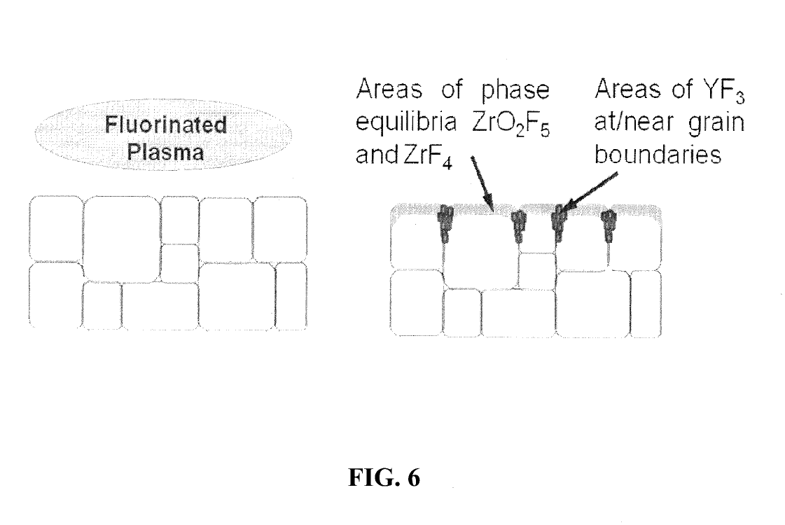

The activated surface may be continuous or discontinuous. For example, in certain embodiments, there may be “islands” of fluoride phases in a surface comprising an oxyfluoride phase. For example, the fluorinated metal oxide surface may comprise a mixture of metal oxyfluoride and metal fluoride phases. FIG. 6 shows an example of an activated surface having oxyfluoride and fluoride phases.

The thickness of the activated surface may vary. For example, in certain embodiments, the activated surface has a thickness of from about 0.5 nm to about 5 nm, preferably from about 1 nm to about 5 nm, and more preferably from about 1 nm to about 3 nm. In certain aspects, this means that the medical implant comprises a fluorinated metal oxide surface having a thickness within these ranges.

The fluoride-treated surface of the medical implant may be further functionalized with a silane coupling agent as described above to give a medical implant with an organic-functionalized surface. A cement may be reacted with the silane coupling agent to give a medical implant coated with cement, which may be subsequently affixed to a variety of surfaces. In one embodiment, the cement is covalently bonded to the silane coupling agent. In one embodiment, the medical implant is a dental component that may be affixed using the cement to any natural or synthetic dental component or substrate.

The fluoride-treated surface of the medical implant may alternatively be directly functionalized with a cement, giving a medical implant coated with cement. In one embodiment, the cement is covalently bonded to the fluoride-treated surface. In certain embodiments, the cement is bonded more strongly to the fluoride-treated surface than cement bonded to an untreated medical implant.

In one embodiment, the fluoride-treated implant is coated with a silicon oxide layer using molecular vapor deposition. The coating may be full or partial. The coating may be continuous or discontinuous. In one embodiment, the coating is full, meaning that the surface of the medical implant is completely coated. The thickness of the coating may vary. In some embodiments, the silicon oxide coating is chemically attached or chemisorbed to the fluoride-treated surface of the medical implant. By chemically attached or chemisorbed is meant that there exists a chemical bond between the silicon oxide coating and the fluoride-treated surface of the medical implant. The bond may be of any strength and type, but is preferably a strong covalent bond. In some embodiments wherein multiple layers of silicon dioxide are deposited, the additional layers may be physisorbed onto adjacent layers rather than chemisorbed. In such embodiments, the silicon oxide coating may be further functionalized with a silane coupling agent as described above to give a medical implant with an organic-functionalized surface. A cement may be reacted with the silane coupling agent to give a medical implant coated with cement, which may be subsequently affixed to a variety of surfaces. In one embodiment, the cement is covalently bonded to the silane coupling agent. In one embodiment, the medical implant is a dental component that may be affixed using the cement to any natural or synthetic dental component or substrate.

Although the description and examples focus on dental components as an example of medical implants, the methods and compositions of the invention may also be applicable to other types of medical implants. For example, orthopedic implants such as replacement joints which are affixed by a cement may be treated according to the methods of the present invention.

### EXPERIMENTAL

### Example 1

### Zirconia Surface Modified by Fluorination, Bonded to Organosilane and Then to Cement Materials and Methods

Blocks of pre-sintered zirconia (ZirCAD®, Ivoclar-Vivadent, Schaan, Liechtenstein) measuring 14×12×20 mm were obtained from the manufacturer and sectioned into 2 mm plates. Composite cylinders (Filtek™ Supreme, 3M-ESPE™, St. Paul, Minn.) were fabricated by condensing the material into a Teflon mold (2 mm diameter×3 mm height) and UV light-activated for 40 seconds at 500 mW/cm². Surfaces of each material were highly polished through 50 μm diamond grit polishing paper to ensure starting surface roughness. After polishing, select surfaces were air-abraded (50 μm alumina abrasive, 0.29 MPa, 20 sec) prior to chemical surface treatments and/or bonding procedures. Abraded specimens were rinsed with iso-propanol and submersed in DI ultrasonic bath for 5 minutes.

Zirconia specimens were fluorinated in a planar, inductively coupled 13.56 MHz plasma reactor at 800 W with a dc bias of −300V. A continuous flow source gas of SF₆ at 25 sccm was used to maintain a pressure of 35 mT for 2 min. X-ray photoelectron spectroscopy (XPS) was used to evaluate surface chemistry and stoichiometry of the conversion layer. A Kratos Analytical Axis Ultra XPS system with a monochromatic Al k␣ source operated at 15 kV and pass energy of 20 eV was used to obtain Zr 3d core level spectra. The spectra was then deconvoluted using CasaXPS™ software employing a Shirley background subtraction and mixed Gaussian-Lorentzian (G-L) peaks associated with the oxide and oxyfluoride components. The spectra were referenced to the Zr 3d_{5/2} peak at 182.2 eV for ZrO₂.

Below are the seven groups (n=10) from which shear bond specimens were fabricated, with variations for each surface treatment. All shear bond specimens were prepared using the same bonding procedure. Zirconia surfaces were modified (see below for modification techniques) and treated with an organosilane (Monobond-S, Ivoclar-Vivadent, Schaan, Liechtenstein) prior to resin cement bonding. Composite cylinders were coated with resin cement (Rely-X™ Unicem, 3M-ESPE™, St. Paul, Minn.), placed on the zirconia surface, and UV-light cured under a defined load (5 N):
Group 1 and 2: (control): (1) Polished, untreated surface and (2) roughened, untreated surface.
Group 3 and 4: Surfaces were polished (3) or roughened (4) and were modified with a 3 nm SiₓO_{y} layer (this procedure is described in detail in J. R. Piascik et al., Surface Modification for Enhanced Silanation of Zirconia Ceramics, Dental Mater. 25: 1116-1121(2009), incorporated herein by reference in its entirety). Group 5: Zirconia surfaces were silica-coated using 30 μm alumina particles modified with salicylic acid (CoJet®, 3M-ESPETm, St. Paul, Minn.-0.28 MPa, 5-10 mm working distance, 15 sec).

Group 6 and 7: Zirconia surfaces were polished (6) or roughened (7) and were exposed to the fluorination process described above.

Shear bond test specimens were stored in DI water at 37° C. for a period of 24 hours prior to testing. Specimens were then fixed to a custom vise fixture to ensure vertical compliance. All specimens were subjected to a force at a crosshead speed of 0.5 mm/min in an electro-mechanical testing device (Instron Corp, Norwood, Mass.). Shear bond strengths were calculated by dividing peak load by the cross-sectional area of the composite cylinder. Single-factor analysis of variance (ANOVA) at a 5% confidence level was performed for the bonding strength data. Optical microscopy and scanning electron microscopy (SEM) were used to evaluate and quantify failure surfaces.

### Results

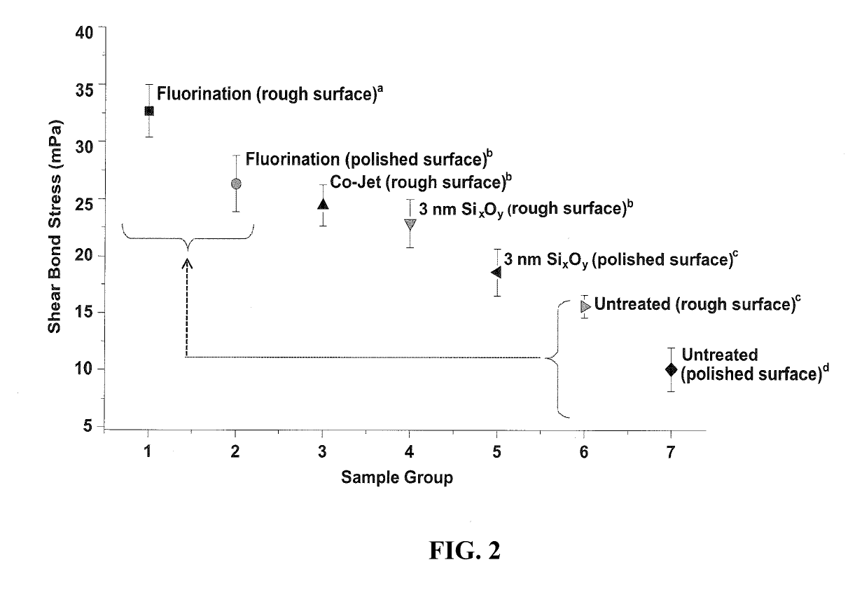

The mean values and standard deviations of the shear bond strength mechanical testing are graphically shown in FIG. 2. In this figure, shear bond stress values for all groups tested are provided, with values plotted with standard deviation error bars (brackets { } denotes effect of fluorination process on roughened and polished surfaces respectively). It should be noted that the fluorinated (polished) group was statistically the same as a clinically accepted tribochemical treatment. Single-factor ANOVA analysis revealed a significant difference in mean shear bond strengths. As expected, the untreated polished zirconia specimens were shown to have the lowest shear strength. The fluorinated zirconia specimens (both rough and polished) displayed the highest shear bond strengths as compared to other commercially available treatments. Furthermore, the fluorinated polished specimens were statistically similar to those that were mechanically roughened using a commercial tribochemical approach.

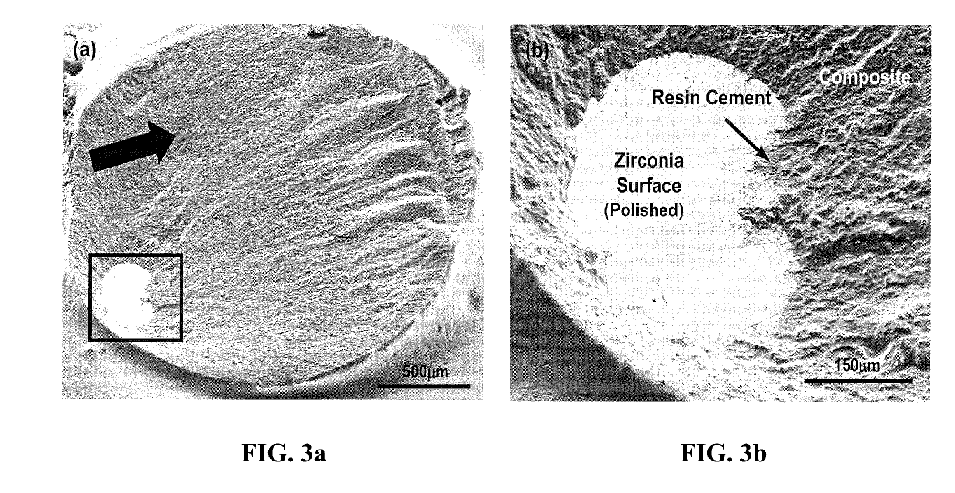

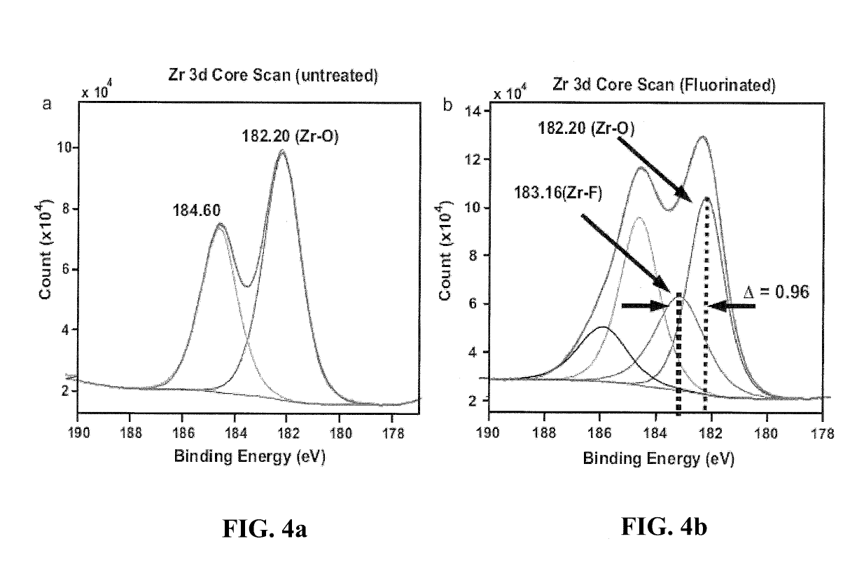

Table 1, below, displays the shear bond values with standard deviation and percent failure mode. Optical and SEM analyses revealed a higher percentage of adhesive/cohesive failures for the fluorinated group of specimens. This type of failure indicates high bond strength between the two substrates due to the nature of shear bond testing. The force placed on the cylinder during testing creates a dual-mode of tensile and compressive stresses at the bonding interface, thus creating a failure surface that reveals an area of adhesive failure (noted by exposure of either zirconia surface and/or resin cement) along with composite still adhered to the zirconia surface. There are several factors that can contribute to variations in shear bond load values. Larger bonding areas can induce processing flaws which can promote premature bond failure, and variation in bonded composite can generate disparities in shear bond values. FIG. 3 shows representative SEM micrograph images of a fluorinated (polished) specimen with adhesive/cohesive failure. FIG. 3(a) is a low magnification where the arrow shows shear force direction and 3(b) is a high magnification area within the box. The white areas are the zirconia surface and small dark regions shows areas of resin cement and composite.

TABLE 1
Shear bond stress (MPa) with standard deviation
of the different test groups.

| Sample Group | Shear Bond Stress | Standard | A | A/C |
|---|---|---|---|---|
| (with surface finish) | (MPa) | Deviation | (%) | (%) |
| Fluorination (rough)^{a} | 32.67 | 6.43 | 10 | 90 |
| Fluorination (polished)^{b} | 26.32 | 6.35 | 30 | 70 |
| Co-Jet ™ (rough)^{b} | 24.44 | 4.94 | 30 | 70 |
| 3 nm SiₓO_{y} (rough)^{b} | 22.88 | 4.69 | 40 | 60 |
| 3 nm SiₓO_{y} (polished)^{c} | 18.58 | 2.79 | 80 | 20 |
| Untreated (rough)^{c} | 15.58 | 1.98 | 90 | 10 |
| Untreated (polished)^{d} | 10.08 | 3.76 | 100 | 0 |

The A column shows the percent of samples displaying adhesive failure; the A/C column shows the percent of samples displaying a mixed mode of adhesive and cohesive failure. The superscripted letters in the first column represent the same statistical grouping (i.e., items with the same letter are statistically the same).

XPS survey scans of an untreated specimen were used to establish a baseline of near surface chemistry for comparison to fluorination results. Survey scans of the fluorinated specimens revealed the presence of fluorine (is) accompanied by a reduction in oxygen (is). XPS Core scans of the Zr⁺ 3d doublet as shown in FIG. 4 were performed on an untreated zirconia specimen (FIG. 4(a)) and a fluorinated zirconia specimen (FIG. 4(b)). These scans highlighted an interesting phenomenon: In addition to the Zr—O doublet at 182.20 eV, there was a component of the signal shifted to higher binding energy, 183.16 eV (see FIG. 4(b)). This increase in binding energy suggests a structure that is more ionic (and more reactive) and characteristic of zirconium oxyfluoride (ZrOₓF_{y}).

### Discussion

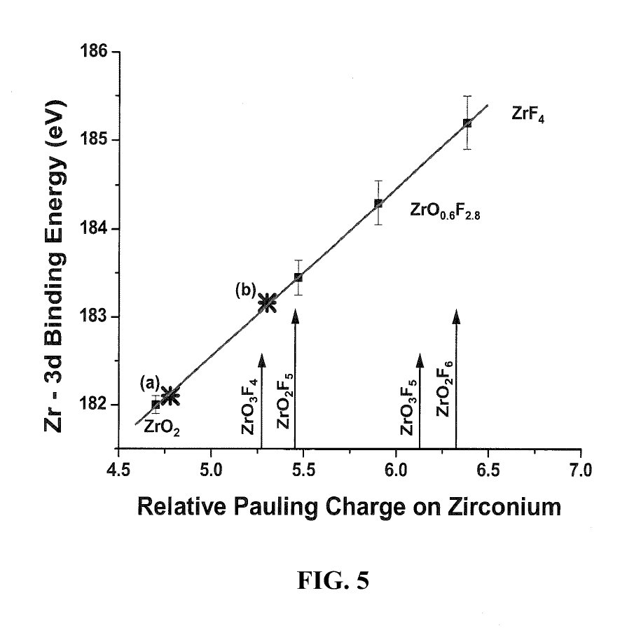

Based on an article by Pantono and Brow (J Am. Ceram. Soc. 1988; 71(7): 577-581), incorporated herein by reference in its entirety, we can approximate the zirconium oxyfluoride stoichiometry for the above specimen as, ZrO₃F₄ (see FIG. 5). FIG. 5 shows the relationship between Zr 3d binding energy and the Pauling charge on Zr⁺-cation. Added data points are the Zr 3d binding energies measured via XPS from an (a) untreated and (b) fluorinated specimen, respectively.

To determine depth and chemical bonding modification to the structure, angle resolved and Ar-sputter XPS were performed. Based on these experiments and grain size analysis of the zirconia, it is proposed that the fluorination treatment converts the surface of ZrO₂ to a zirconium oxyfluoride with an average thickness of 20-30 Å and non-uniformly distributed across the surface. See FIG. 1 for a schematic representation of the sintered zirconia and the subsequent oxyfluoride conversion. Fluorinated plasma is applied to the zirconia surface, converting the top 1-3 nm into a surface comprising zirconium oxyfluoride (ZrOₓF_{y}). The oxyfluoride surface can react with organosilanes, enabling silicon attachment to the surface.

In order to test adhesion strength of dental materials, simple shear bond or microtensile mechanical testing is often used. Both, however, have drawbacks when attempting to evaluate the true bond strength. It has been reported that microtensile tests are better at eliminating any macro-sized flaws produced when fabricating specimens, thus providing a closer approximation to the ideal strength. Unfortunately these samples are very time consuming to produce and simply cutting the specimens into final form may introduce stresses from the cutting tool that cannot be quantified. Shear bond testing, however can be used as general baseline and a clinically more relevant bonding area. The test does introduce a multi-mode stress profile: the bonding area will experience tensile stresses at the top (initial point of force) and compressive forces near the center and bottom of the bonding interface.

In order to establish an understanding of how surface preparation and bonding procedures are critical to bond strengths, both polished and physically abraded surfaces were evaluated. Relatively low bond strengths (ranging from 10-15.6 MPa for polished and roughened, respectively) are reported here for specimens bonded with a phosphoric acid modified methacrylate monomer cement. These results are not unexpected since other reports have shown that unmodified surfaces display low bond strengths and eventually lead to adhesive failure. The lack of chemical bonding between the two materials is the overriding contributing factor for low bond strengths. This had led to research efforts that seek chemical and mechanical techniques that improve adhesion.

Interestingly, a fluorinated surface, either polished or roughened, displayed the highest shear bond strengths (26.3 and 32.7 MPa, respectively). It is noted that 70% of the fluorinated polished specimens exhibited adhesive/cohesive behavior, whereas 0% of the untreated polished specimens displayed this characteristic. These data show that the fluorinated treatment on roughened zirconia displayed the highest shear bond strength and even more promising is that the fluorinated treatment on polished zirconia was statistically the same as (or higher than) other clinically accepted methods. This finding suggests that the fluorination treatment could be used on as-received substrates, where roughening or other surface modification techniques are neither possible nor desired. Although not wishing to be bound by theory, it is hypothesized that the presence of an oxyfluoride phase on the zirconium oxide surface may increase its reactivity with silanes by facilitating Zr-hydroxylation via H—F extraction in the presence of water. Oxyfluorides have been shown to be more reactive in aqueous environments.

### Conclusion

Simple shear bond mechanical tests demonstrated that a fluorination pre-treatment is a viable method to chemically modify zirconia to produce a reactive surface for adhesive bonding. By using

XPS analysis, it was determined that this novel treatment process created an oxyfluoride conversion layer that is receptive to organosilane chemical attachment.

### Example 2

### Yttria-Stabilized Zirconia Modified by Fluorination and Bonded Directly to Cement

Presented in this example is an in-depth analyses of the fluorination process on YSZ surfaces and the resulting phases that form in the thin conversion layer (see, for example, FIG. 6). The motivation for this work was to create a reactive surface that would allow for chemical interaction with acrylate based resin cement without the use of silanes or primers. Simple shear bond tests were employed to measure adhesion on as-received (non-roughened) and roughened specimens and compared to alternative pretreatment techniques.

### Materials and Methods

Pre-sintered plates and cylinders of YSZ shear bond specimens (LAVA, 3M ESPE AG; Seefeld, Germany) were obtained from the manufacturer. As-received surfaces (both plates and cylinders) were air-abraded (50 μm alumina abrasive, 0.29 MPa, 20 sec) prior to surface modification treatments and rinsed with isopropanol, then ultrasonically cleaned in DI for 5 minutes. Bonding surfaces were then fluorinated in a planar, inductively coupled 13.56 MHz radio-frequency plasma reactor at 800 W with a dc bias of ˜300V. Water cooling of the substrate platform ensured process temperatures did not exceed 100° C. A continuous flow source gas of SF₆ at 25 sccm was used to maintain a pressure of 35 mT at varying times of 20 sec, 2 min, and 5 min. For each process time, YSZ cylinders (n=12) were coated with resin cement (Rely-X Unicem, 3M-ESPE, St. Paul, Minn.) per manufacturer's instructions, placed directly on the plate surface and UV-light-curing was performed under a defined load (5 N). Untreated specimens were used as a control for the shear bond testing. Shear bond test specimens were stored in DI water at 37° C. for a period of 24 hours prior to testing, then fixed to a custom fixture to ensure vertical compliance. Specimens were subjected to a force at a crosshead speed of 0.5 mm/min in an electro-mechanical testing system (Instron Corp, Norwood, Mass.). Shear bond strengths were calculated by dividing peak load by the cross-sectional area of the composite cylinder. Single-factor analysis of variance (ANOVA) at a 5% confidence level was performed for the bonding strength data for statistical similarities. Scanning electron and optical microscopy was used to evaluate bonding surfaces.

X-ray photoelectron spectroscopy (XPS) was used to evaluate surface chemistry and stoichiometry of the conversion layer. A Kratos Analytical Axis Ultra XPS system (Manchester, UK) with a monochromatic Al kα source operated at 15 kV and pass energy of 20 eV was used to obtain surface survey, Zr and Y 3d core level spectra and deconvoluted using CasaXPS™ software. A Shirley background subtraction and mixed Gaussian-Lorentzian (G-L) peaks associated with oxide, oxyfluoride, and fluoride components were deconvolved to reveal near surface phases operative in adhesive bonding chemistry. Additionally, as-received YSZ plates were exposed to the above mentioned fluorination times. X-ray diffraction (XRD) (Philips X'Pert PRO MRD HR, PANalytical Inc., Westborough Mass.) was used to quantify potential phase transformation post exposure.

### Results

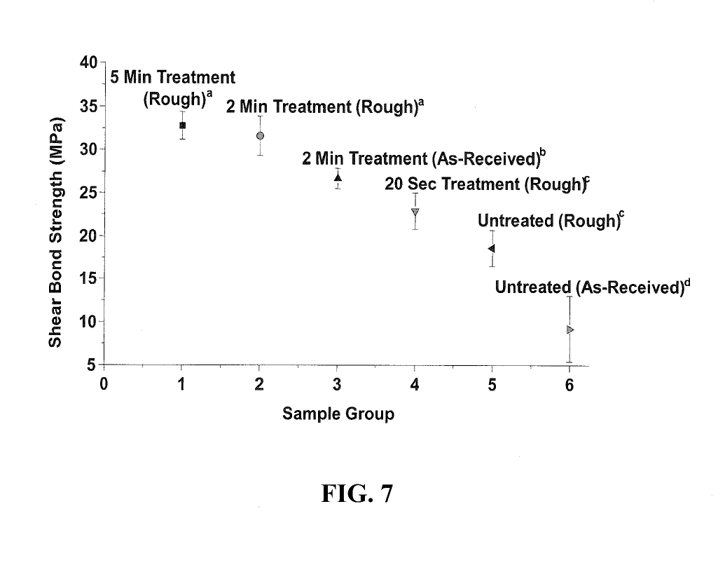

Shear bond values of all groups tested are shown graphically in FIG. 7 with standard error bars. The shear bond data show an increase in bond strength with treatment time. As expected the as-received (polished) specimens displayed lowest bond strengths, indicating no chemical attachment between YSZ surfaces and resin cement. As-received, untreated, and 2 min fluorinated specimens were tested to evaluate potential non-roughening effects on adhesive strength. It should be noted that the 2 min as-received (polished) group was statistically higher as compared to the clinically accepted method (roughened +resin cement). The 5 and 2 minute treated specimens were shown to have the highest bond strengths, 33.7 and 31.5 MPa, respectively. Table 2 displays shear bond strength values with standard deviations. The superscripted letters in the first column represent the same statistical grouping (i.e., items with the same letter are statistically the same).

TABLE 2
Shear bond stress (MPa) with standard
deviation of the different test groups

| | | Standard |
|---|---|---|
| Sample Group | Shear Bond Stress | Deviation |
| (w/ surface treatment) | (MPa) | (%) |
| 5 minute treatment (rough)^{a} | 33.7 | 6.4 |
| 2 minute treatment (rough)^{a} | 31.5 | 6.9 |
| 2 minute treatment (as-received)^{b} | 26.7 | 4.9 |
| 20 second treatment (rough)^{c} | 22.9 | 4.7 |
| Untreated (rough)^{c} | 18.6 | 2.8 |
| Untreated (as-received)^{d} | 9.2 | 6.2 |

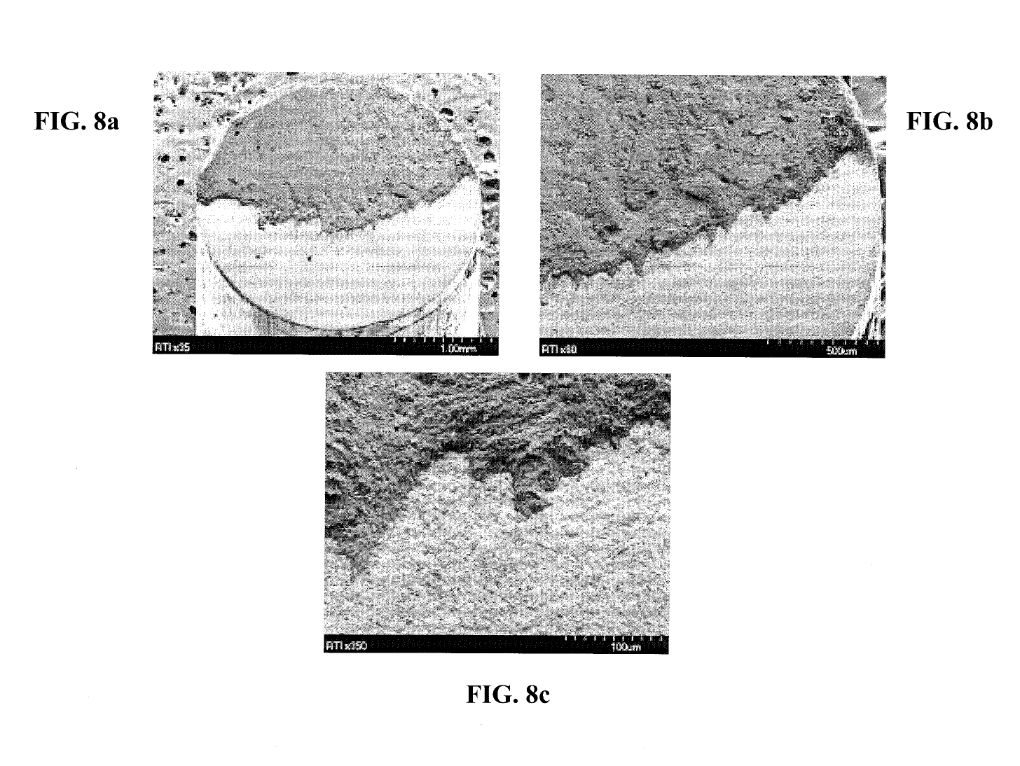

Evaluation of failure modes differ from conventional shear bond analysis due to the fact that shear bond specimen components were the same material. Typically, when testing two dissimilar materials, with distinct differences in material properties, it would be common to see either an adhesive failure, cohesive, or mixed mode (failure displaying both adhesive and cohesive properties). Here, all failures are quantified as adhesive failures (see FIG. 8), due to the fact that resin cement is the weak link in the bonding of the two materials. All failure surfaces, with the exception of the untreated groups, displayed a percentage of resin cement bonded to both plate and cylinder. Based on the shear bond values, there are two scenarios that can be considered for the increase in bond strength: (1) an increase in surface area due to particle air-abrasion as shown in as-received compared to roughened groups and/or (2) the increase in surface reactivity with the resin cement facilitating increase in covalent bonding between the substrate and cement.

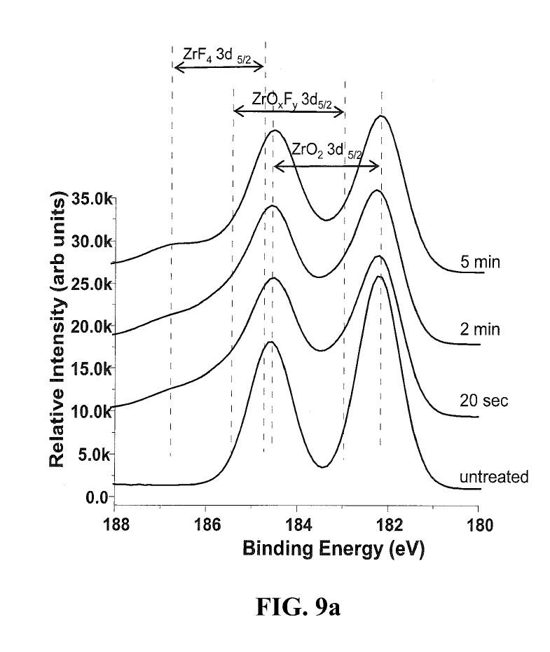

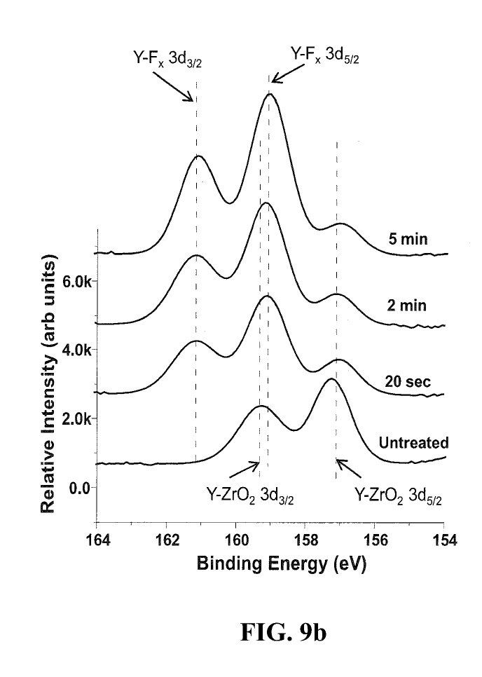

X-ray photoelectron spectroscopy (XPS) analysis was performed on both as-received (non-roughened) and fluorinated YSZ plates, and used to evaluate the chemistry and stoichiometry of the conversion layer. All spectra were referenced to the Zr 3d_{5/2} peak at 182.2 eV for ZrO₂. FIG. 9A and 9B show Zr and Y 3d spectra, respectively, as a function of fluorination time (referenced to unprocessed YSZ). The deconvolved spectra reveal formation of Zr-oxyfluoride, Zr-fluoride, and Y-fluoride for process durations of 20 sec to 5 min. The Zr 3d spectra were characterized by similar proportional amounts of oxyfluoride and fluoride phases. The near surface yttrium levels, however, increased considerably with fluorination time, and by as much as 54% for the 5 min specimen (relative to the unprocessed YSZ). This result is noteworthy and will be discussed in greater detail in the following sections. Furthermore, yttrium fluoride (YF) was observed, and increased with longer processing, and in contrast to the zirconium phases, no evidence of Y-oxyfluoride phases were detected. The collective data indicate a broad processing window for producing a reactive surface conversion layer.

XPS revealed an increase in both fluoride and oxyfluoride compounds on the surface of treated specimens. Interestingly, shear bond strength and change in % Y surface concentration trend in the same direction as a function of treatment time (FIG. 10(a)). Data showed that as surface treatment time increased, so did adhesion strength and % Y concentration. The increase in bond strength would indicate that the surface is becoming populated with a higher concentration of reactive sites leading to an increase in potential covalent bonding with the resin cement. Simple contact angle measurements, sessile drop method, were performed to evaluate the wettability of a planar untreated and a 2 min fluorinated specimen. The contact angle for the untreated specimen is 58° (FIG. 10(c)) and a specimen after a 2 min treatment is 6° (FIG. 10(b)). This change to a lower contact angle would indicate a surface that is highly hydrophilic, increasing its wettability and surface reactivity.

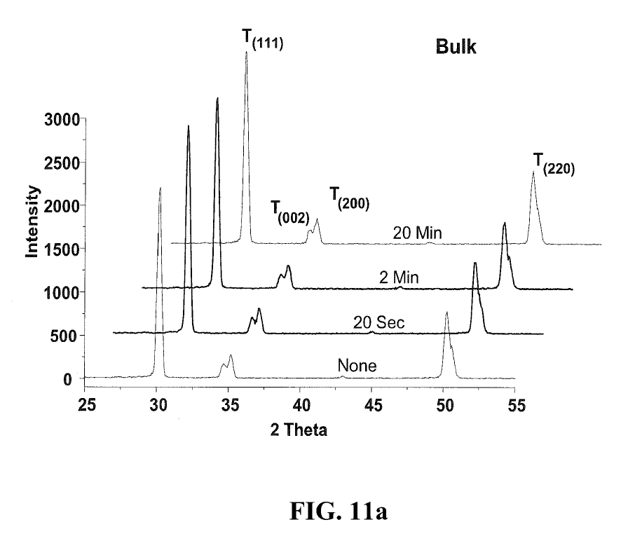

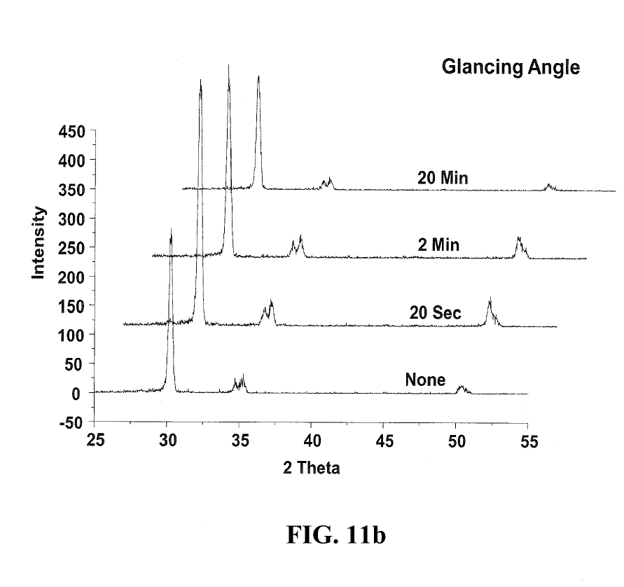

X-ray diffraction was performed on the above mentioned specimens to evaluate crystal structure and potential phase transformation (tetragonal to monoclinic) based on treatment time. FIG. 11A displays diffraction 2-theta scans revealing that YSZ untreated and treated specimens consist of purely tetragonal phases. No monoclinic phases were detected within the resolution of the diffractometer, suggesting that the fluorination plasma treatment used in this study will not elicit a tetragonal to monoclinic phase transformation. Based on the fact that this was a surface treatment, limited to the top 2-5 nm, glancing angle)(˜2° diffraction was also performed (FIG. 11B). These results mirrored the bulk diffraction analysis, indicating that the crystal structure, even near the surface, is tetragonal and apparently unchanged from that of the as-received bulk material.

### Discussion

This study evaluated an alternative method to increase the wettability and chemical reactivity of YSZ surfaces using a novel fluorination technique. It has been well established that application of silane primers to silicon-based materials show increased adhesion with resin cements and bond to surface hydroxyl groups of polar surfaces. However, these techniques used by clinicians are not suitable for zirconia-based materials, which are classified as inert or non-reactive.

As controls for the present study, untreated polished and roughened specimens were evaluated for adhesion. The polished specimens displayed the lowest strength (9.2 MPa), as expected and in agreement with the previous example. Surface roughening increased the strength (18.6 MPa); however, this is attributed primarily to an increase in surface area and not to chemical attachment with the resin cement. As shown in FIG. 10(a), an increase in fluorination time resulted in increasing shear bond strength, suggesting that there may be further optimization of this treatment that could potentially exhibit higher bond strengths, a more robust interface, or some combination of the two. It is also noted that there is a saturation in the trend and more detailed chemical analyses are currently underway to understand the nature of this trend. As shown earlier, this increase in bond strength suggests that the enhanced surface reactivity may be directly correlated to the conversion of the Y—ZrO₂ (YSZ) structure to three distinct phases of Zr-oxyfluoride, Zr-fluoride, and Y-fluoride.

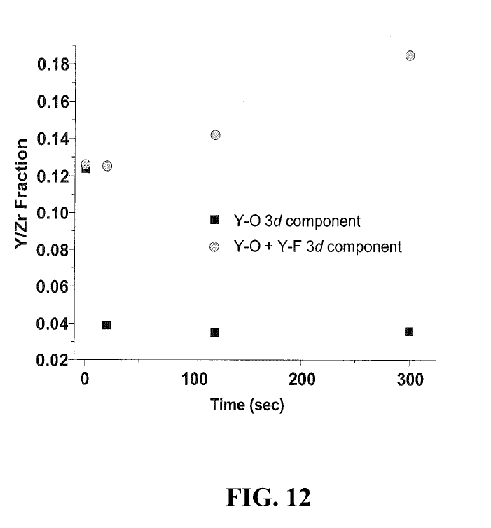

To help explain the reactivity of fluorinated zirconia and yttria compounds, we recalled an earlier study by Pantano and Brow (J. Am. Ceram. Soc. 1988; 71(7): 577-581), which investigated the surface reactivity associated with hydrolysis of fluorizirconate glasses. They used XPS to characterize the various stoichiometries of zirconium oxyfluorides (ZrOₓF_{y}) by plotting the binding energy for the Zr 3d photoelectron as a function of the Pauling charge on the Zr⁺-ion. It was reported that subsequent Zr-oxyfluoride phases produced during hydrolytic exposure are seven-fold coordinate species. The seven-fold coordination is based on one fluorine loss for each oxygen incorporated into the oxyfluoride phase. A detailed surface analytical study of the plasma fluorination of YSZ confirmed that the phases present at the surface are of 7-fold symmetry and propose that the Zr-oxyfluoride stoichiometry formed during this plasma conversion process is ZrO₂F₅. Furthermore, in comparing the relative Yttrium to Zirconium levels as a function of fluorination time (FIG. 12) and then assigning their bonding components via XPS deconvolution, we discover that there is an increase in total Yttrium concentration at the surface, and that increase is primarily associated with the formation of yttrium fluoride (YF₃).

Low temperature (<100° C.) diffusion of Y in YSZ has not been reported; however, the data in FIG. 12 show a greater than 50% increase in Y/Zr ratio within the top 3-5 nm after 20 min. of plasma fluorination. The XPS deconvolution attributed this increase to YF₃ formation. It is possible that this increase in Y-surface concentration could be the result of grain-boundary depletion and surface diffusion, driven by the strong chemical potential formed by the presence of fluorine on the surface. In addition to forming a Zr-oxyfluoride phase, the majority of the original Y-ZrO₂ and increased yttrium appears to be converting to Y-fluoride. One concern was that significant depletion of Y from the sub-surface YSZ lattice might drive the metastable tetragonal lattice towards the room temperature monoclinic phase. However, the surface conversion layer is only 3-5 nm thick and data from glancing angle x-ray diffraction (FIG. 11B) detect only tetragonal phases. The source of this increased Y/Zr ratio in the near surface region and the role it plays in increasing surface reactivity is the subject of ongoing research. Although not bound by any particular theory of operation, it is believed that the zirconium-oxyfluoride phase is the dominant contributor to increased bond-strength for YSZ surfaces and future work will involve studying the chemical bonding between this surface and various acrylate compounds and the roles that conversion layer thickness and stoichiometry play on resulting bond strength and phase stability.

### Conclusion

This study analyzed YSZ to YSZ adhesion using a common acrylate-based resin and mechanical data revealed an increase in adhesion strength as a function of fluorination exposure time. This modification process did not utilize an organosilane coupler or metal primer to increase chemical bonding between the substrates, potentially eliminating the need for silanation. It is hypothesized that these results can be applied to other bonding scenarios involving YSZ (i.e., composites, titanium, porcelain, etc.). XPS analysis revealed an increase in Y-fluoride, as well as Zr-oxyfluoride and Zr-fluoride with treatment time.

1.-16. (canceled)

17. A method of preparing the surface of a medical implant, comprising:
providing a medical implant comprising a substrate surface formed of a material comprising available hydroxyl groups; and
treating the medical implant with a fluorine-containing reagent to provide a fluorinated metal oxide on the implant surface.

18. The method of claim 17, wherein the substrate surface comprises zirconia, alumina, titania, chromium oxide, or a combination thereof.

19. The method of claim 17, wherein the medical implant comprises one or more dental components selected from the group consisting of dental implants, crowns, bridges, fillings, veneers, inlays, onlays, endodontic devices, and orthodontic brackets.

20. The method of claim 17, wherein the medical implant is a dental component and the method further comprises reacting the implant surface having the fluorinated metal oxide thereon with a silane coupling agent.

21. The method of claim 20, wherein the silane coupling agent is selected from the group consisting of 3-methacryloyloxypropyltrimethoxysilane, 3-trimethoxysilylpropylmethacrylate, 3-acryloyloxypropyltrimethoxysilane, 3-isocyanatopropyltriethoxysilane, N-[3-(trimethoxysilyl)propylethylenediamine], 3-mercaptopropyltrimethoxysilane, bis-[3-(triethoxysilyl)propyl]polysulfide, and combinations thereof.

22. The method of claim 20, further comprising coupling the silane coupling agent to a dental cement.

23. The method of claim 17, wherein the medical implant is a dental component and the method further comprises reacting the implant surface having the fluorinated metal oxide thereon with a dental cement, with no silane coupling agent therebetween.

24. The method of claim 22, wherein the dental cement is selected from the group consisting of polymer-based adhesives, cements and composites, resin-modified glass ionomers, and combinations thereof.

25. The method of claim 22, further comprising bonding the dental component to a second dental component with the dental cement.

26. The method of claim 25, wherein the surface of the second dental component is constructed of a material selected from the group consisting of natural tooth, metal, porcelain fused to metal, porcelain, ceramic, resin, and combinations thereof.

27. The method of claim 17, wherein the treating step comprises plasma treatment.

28. The method of claim 17, wherein the treating step comprises physical roughening or chemical etching of the implant surface prior to or at the same time as treating the implant with the fluorine-containing reagent.

29. The method of claim 17, wherein the fluorine-containing reagent is sulfur hexafluoride (SF₆).
